(19) United States
(12) Patent Application Publication
(10) Pub. No.: US 2019/0126003 A1
(43) Pub. Date: May 2, 2019

(54) METHODS AND COMPOSITIONS FOR TREATING EMOTIONALLY BASED DYSREGULATION IN MENTAL HEALTH DISORDERS

(71) Applicant: CJT, LLC, Potomac, MD (US)

(72) Inventor: Susan A. BODANSKY, Potomac, MD (US)

(21) Appl. No.: 16/175,315

(22) Filed: Oct. 30, 2018

Related U.S. Application Data
(60) Provisional application No. 62/578,799, filed on Oct. 30, 2017.

(51) Int. Cl. A61M 21/02; A61H 9/00; A41D 1/04; A63B 21/065

(52) U.S. Cl. CPC A61M 21/02; A61H 9/0092; A61M 2021/0022; A63B 21/065; A41D 1/04

(57) ABSTRACT

The invention encompasses methods and compositions for treating and preventing emotionally based dysregulation in various mental health and emotional disorders by among other things applying deep pressure to assist in feeling grounded (relaxed, present and with a strong sense of self) and decrease anxiety in the subject. The methods and compositions include a wearable device constructed for covering at least a portion of the torso of a subject. The wearable device includes individually expandable units for applying pressure to a specific body region of the individual when expanded and an expandable support unit for expanding and countering the pressure applied to the specific body region by each individual expandable pressure unit.

### FIELD OF THE INVENTION

The invention encompasses methods and compositions for treating and preventing emotionally based dysregulation by among other things applying deep pressure to a subject. The methods and compositions include a wearable device constructed for covering at least a portion of the torso of a subject. The wearable device includes individually expandable units for applying pressure to a specific body region of the individual when expanded and a plurality of compartments for including a weighted object.

### BACKGROUND OF THE INVENTION

Individuals suffering from Autism Spectrum Disorders (ASDs), as well as other behavioral disorders, are oftentimes characterized as having sensory abnormalities.

Treatment of such individuals attempts to lessen the deficits and abnormal behaviors associated with such disorders, and to increase the quality of life and functional independence of these individuals.

One treatment approach involves a manual application of deep pressure to the body of the individual. Temple Grandin [Journal of Child and Adolescent Psychopharmacology 2 (1) 1992] postulated that deep pressure can serve to organize the central nervous system and modulate the sensory stimuli, and that this in turn leads to a temporary decrease in tactile defensiveness resulting in a decrease in physiological arousal.

### SUMMARY OF THE INVENTION

The invention encompasses methods and compositions for treating and preventing emotionally based dysregulation in various mental health and emotional disorders by among other things applying deep pressure to activate and relax subject.

In one embodiment, the invention encompasses a wearable device for treating or preventing emotional dysregulation in mental health disorders (e.g., an autism spectrum disorder) in a subject comprising an expandable device covering at least a portion of a torso of a subject comprising: (a) one or more compartments capable of receiving a weighted object; (b) one or more expandable compartments capable of being inflated to apply pressure to a specific body region of the torso of the subject when expanded; c) a mechanism for expanding said individually expandable compartments; and d) a control unit capable of operating said individually expandable units in a sequence of expansions and contractions so as to treat or prevent emotional dysregulation in mental health disorders (e.g., an autism spectrum disorder).

In certain embodiments, the invention further comprises one or more sensing units for measuring at least one physiological parameter of the subject.

In certain embodiments, the sensing unit includes at least one sensor selected from the group consisting of a heart rate sensor, a body temperature sensor, a skin moisture sensor, a muscle activity sensor, a blood pressure sensor, a respiratory sensor and a brain activity sensor.

In certain embodiments, the expandable units apply one or more expansions and contractions determined by physiological parameter data values of the individual monitored by said sensing unit.

In certain embodiments, the wearable device is configured as a vest.

In certain embodiments, the mechanism for expanding includes a manual air pump.

In another embodiment, the invention encompasses a method for treating or preventing emotional dysregulation in a mental health disorder (e.g., an Autism Spectrum Disorder) in a subject comprising providing a subject with a wearable device comprising an expandable device covering at least a portion of a torso of a subject comprising: (a) one or more compartments capable of receiving a weighted object; (b) one or more expandable compartments capable of being inflated to apply pressure to a specific body region of the torso of the subject when expanded; c) a mechanism for expanding said individually expandable compartments; and d) a control unit capable of operating said individually expandable units in a sequence of expansions and contractions so as to treat or prevent emotional dysregulation in mental health disorders (e.g., an autism spectrum disorder)

In certain embodiments, the method further comprises one or more sensing units for measuring at least one physiological parameter of the subject.

In certain embodiments, the sensing unit includes at least one sensor selected from the group consisting of a heart rate sensor, a body temperature sensor, a skin moisture sensor, a muscle activity sensor, a blood pressure sensor, a respiratory sensor and a brain activity sensor.

In certain embodiments, the expandable units apply one or more expansions and contractions determined by physiological parameter data values of the individual monitored by said sensing unit.

In certain embodiments, the wearable device is configured as a vest.

In certain embodiments, the mechanism for expanding includes a manual air pump.

### DETAILED DESCRIPTION OF THE INVENTION

The invention encompasses generally wearable devices capable of applying pressure and weight to the torso of an individual.

As referred to herein, the term “subject” refers to a human subject (e.g. human adult or child) who is suffering from anxiety, stress or a disorder such as emotional dysregulation.

In certain embodiments, the wearable device is capable of applying pressure and weight to the torso of the individual when worn and activated. The wearable device can be configured as a vest, shirt, coat or any other configuration (e.g. torso band) capable of applying pressure.

In certain embodiments, pressure can be activated manually by the human subject or activated according to input from sensing (e.g., physiological sensors) and control units. In certain embodiments, the control unit is capable of controlling the application of pressure according to sensed physiological parameters and/or preselected pressure application sequences, which are triggered by physiological sensor data or selected at random. In certain embodiments, pressure can be applied via mechanical expandable units.

In certain embodiments, the wearable device includes expandable units that are expendable using air.

In certain embodiments, the force applied by the body on the expandable unit combined with the weight can displace the expandable unit away from the body and reduce the force applied thereby on the body region. A vest or coat configuration tightly bound around the body via straps and the like can counteract such forces and displacement, however, tightly binding the wearable device around the body can also reduce the effect of therapy.

In certain embodiments, a wearable device is configured as a vest having one or preferably several individually expendable units, which are preferably actuated (inflated/deflated) using air, and/or one or more compartments for a weighted object. In certain embodiments, units are fabricated from a non- or semi compliant polymer such as PVC and are shaped as flat (substantially two dimensional) squares, rectangles (shown), circles or ovals when empty. When filled with a fluid, expandable units inflate and assume a three dimensional shape that protrudes from a plane defined by the base of each unit. The three dimensional shape can have a height of 10-60 cm and a volume of about 1-5 liter.

Units are capable of supporting an internal fluid pressure of up to 0.1-1.0 Bars when filled.

In certain embodiments, the wearable device can be arranged to contact the lower back, the sides of the back, and shoulders/chest of the subject when the wearable device is worn by the subject.

In certain embodiments, the wearable device includes an expandable compartment, which serves to counter the pressure applied to a specific body region by unit. The expandable compartment is fabricated from a non- or semi compliant polymer such as PVC and is shaped as flat (substantially two dimensional) structure when empty. When filled with a fluid, expandable compartment inflates and assumes a three dimensional shape. The three dimensional shape can have a height of 10-60 cm and a volume of 1-4 liters. In certain embodiments, the expandable compartment is capable of supporting an internal fluid pressure of 0.2-0.5 Bars when filled.

In certain embodiments, inflation of one large inflatable compartment can be used to tightly fasten wearable device to the body and counteract the outward inflation force when expandable compartments are actuated. Such fastening can be effected using a pressure which is high enough to stabilize wearable device on the body when in use.

In certain embodiments, the wearable device can further include waist straps to further stabilize it on the body of the individual. In certain embodiments, waist straps are positioned at a body portion which is not typically subjected to therapeutic pressure and as such would not interfere with treatment.

Notwithstanding, a waist strap having individual inflatable units for providing an extra measure of support is also envisaged herein.

In certain embodiments, the expandable compartments portion of wearable device can be fabricated as a single compartmentalized sheet which can then be covered with a fabric which is provided with fasteners (zippers, Velcro™ straps, buttons etc.) for securing wearable device on the torso. The fabric cover can also be used to store additional components of system.

In certain embodiments, the expandable compartments include actuation of units preferably effected using a pump and a portable power source (battery). In certain embodiments, the expandable compartments include valves positioned at the interface between a pump and conduits.

In certain embodiments, the wearable device includes sensors integrated into wearable device (glued to sheet or fastened to fabric cover) and wired to a control unit (e.g. heart rate electrodes) or they can be attached to a body portion (e.g. moisture sensor attached via adhesive patch directly to skin and wired to control unit).

In certain embodiments, an example of heart rate monitor is as used by joggers worn on their chest; this monitor transmits the data wirelessly (using Bluetooth standard) thus enabling the microprocessor to determine if the stress level is increasing (heart rate increasing) or decreasing (heart rate decreasing).

In certain embodiments, the physiological data monitored by sensors is transmitted to control unit and processed to yield a therapeutic script which activates pump and valves.

The present invention can also be configured for remote monitoring via a communication network (e.g. cellular or Internet). By connecting control unit to a communications-capable device such as a cellular telephone (e.g. Smartphone such as the Apple iPhone) or a computer, data collected thereby can be transmitted to a remote server/device for remote monitoring and possibly control.

As is mentioned hereinabove, the present system can be used to treat normal subjects as well as subjects suffering from a disorder such as PTSD, certain Anxiety Disorders, Attention Deficit Disorders, Autism Spectrum Disorders, etc.

Thus, according to another aspect of the present invention there is provided a method of reducing anxiety in an individual. The method is preferably effected using system as follows. The subject wears the device and activates the wearable device (the device can be turned on by the subject, a care giver locally or remotely). Two modes of operation can be selected: manual—the user or care giver directly controls the inflation/deflation cycles; automatic—control unit controls the operation. No calibration is needed since treatment goals look for trends in values and not absolute values.

Thus, the present invention provides a device system and method for reducing emotional dysregulation disorders in a subject. The wearable device and system of the present invention is advantageous over known devices in that can be secured to the body of the individual without interfering with therapy and it addresses and corrects habituation which can substantially reduce the effectiveness of treatment over time.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

In certain embodiments, the method further includes a Centering Jacket Therapy (“CJT”) comprising a five-step protocol that incorporates the use of a proprietary device and system. CJT is a unique mental health therapy with the intention to foster and teach emotion regulation. The composition and method assists in feeling grounded, (relaxed, present and with a strong sense of self.), to decrease anxiety, increase self-confidence as well as attention/focus. CJT allows the user to master the ability to return to homeostasis. It may also prove to diminish the “freeze” response, dissociation and rumination.

In certain embodiments, CJT is helpful for people who suffer from Post-Traumatic Stress Disorders, certain Anxiety Disorders, Autism Spectrum Disorder, Attention Deficit Disorder, Depressive Disorders, and more. These aforementioned conditions may be characterized by difficulties with emotion regulation, including anxiety, inability to maintain focus, low self-confidence, numbing, and stress. Cognitive Behavioral Therapy has proven to be of some assistance and this treatment uses the CBT strategy of focusing on the relationship between the problem and the thought but in a unique way whereby each step of the protocol anchors to each other in order to foster the mind-body connection. The physiological aspects of CJT connected to positive emotions/thoughts may create an association that fosters and teaches the ability to emotionally regulate. CJT attempts to be inclusive for the individual's unique strengths, interests, and methods of learning. CJT may be used in the Therapist's office, or once trained in use, on one's own (with the ability to self-administer and self-regulate.) It may be used as an educational device to assist the client in experiencing the mind and body connection. CJT may be used at the moment one's “symptoms” or “trauma” are triggered, or it may be used for future recall. After a period of “training time,” it appears that one can “self-regulate” by recalling the Protocol rather than actually going through each step with the assistance of the device.

The composition comprises a “Centering Jacket;” a vest that works through the use of (i) weighted shoulders that may feel like hands, (for adults the weight can vary from 0.5 pounds to over 2 pounds per shoulder,) and (ii) compression. The compression is to be created just at and above the waist (celiac crest.) The compression should be firm but comfortable. (When the individual takes the device off, the compression should have been strong enough that the memory of it is still there.) It is essential that the individual is self-creating the compression by pumping two blood pressure cuffs; the self-creation of the compression is part and parcel of the process of the protocol. At present, the prototype utilizes three blood pressure cuffs that are attached to each other. Once optimized, there may be a single bladder or a double bladder that will facilitate the compression at and above the waist. The vest utilizes a quick release belt that is attached to the vest (not removable,) as a safety precaution. The vest is presently made out of fleece but a more durable material will be utilized once optimized and there may or may not be Velcro and zippers.

In certain embodiments, the invention encompasses a method including five steps incorporating the centering jacket and the following steps:

Step one: Put the Centering Jacket on and self-regulate as well as self-create the compression. Moderate pressure (compression and weight) may be stimulating dermal pressure receptors innervated by vagal afferent fibers leading to a parasympathetic nerve response (reducing anxiety) and cortisol reduction. Feeling contained and in control may also diminish anxiety and increase performance. There may be a synaptic transmission between neurons to remember the comfort of early holding. Moderate pressure may decrease heart rate.

Step two: Take two deep cleansing breaths. Creates intention for the task and assists with memory encoding.

Step three: Guided imagery: Fosters relaxation, creativity, being in control and self-confidence. Reinforces emotional stability and experiencing a feeling of being contained.

Step four: Engaging in a pleasing, positive activity while remaining mindfully in the present. Fifteen minutes with the device on and then another fifteen minutes with the device off. Creates an awareness of the internal and external experience.:

Mindful, present, focused and fun. Mindfulness assists with attention regulation, body awareness emotion regulation and sense of self.

Step five: Reflection.

Integration. Five steps are like a mnemonic device.

Additional benefits: Centering Jacket Therapy is unique, easy to use and consumer friendly. CJT seems to work without having to over-think or spend a great deal of time training in its use. There does not appear to be habituation. It may educate the individual in the understanding and experiencing of the body-mind connection. CJT may promote homeostasis and will hopefully prove mastery of the ability to self-regulate.

CJT, Centering Jacket Therapy, is a mental health intervention/mind-body therapy that incorporates the use of a device. Through clinical trials, CJT facilitates the ability to return to a homeostatic state, and the ability to gain mastery in achieving self-regulation. CJT appears to allow for self-regulation, without habituation. It seems to elicit a feeling of being “grounded” leading to a sense of feeling in control, more self-confident, focused, with increased drive and diminished tension, anxiety and stress. It may also prove to diminish the “freeze” response, dissociation and rumination.

CJT involves a five step protocol, incorporating the use of the device; all steps must be utilized together as no one step is more important than the other and the device remains part and parcel of the entire process. Each step of the Protocol seems to anchor to the others, thereby acting as if a mnemonic device, fostering the body-mind connection and creating an association. It attempts to be inclusive for the individual's unique strengths, including one's senses (sight, hearing, smell and touch,) interests, and methods of learning. This Therapy may be used in the Therapist's office or, once trained in use, on one's own. It may be used as an educational device to assist the client in experiencing the body and mind connection. CJT may be used at the moment one's “symptoms” or “trauma” are triggered, or it may be used for future recall. After a period of “training time,” it appears that one can “self-regulate” by recalling the protocol rather than actually doing it.

Prior to commencing with CJT, a thorough trauma history should be completed and if there is a history of trauma, or any difficulties/injuries with the neck, shoulders or waist, medical approval should be received. The Centering Jacket and the five step protocol may then be appropriately modified and/or adjusted according to the individual's needs. CJT is not to be utilized when experiencing a new trauma or while actively working on trauma with a Therapist as the Protocol is all about tagging to the positive feelings.

CJT builds on aspects of mindfulness, guided imagery, creating intention and neurobiology. It is anticipated that it can facilitate long-term changes in the brain that result in ongoing improved homeostasis/self-regulation, improved mind/body connection and an increased sense of self-confidence in the “self.”

CJT may prove to be helpful for people who suffer from Post-Traumatic Stress Disorder, Attention Deficit Disorder, certain Anxiety Disorders (including performance anxiety,) Autism Spectrum Disorder, Depressive Disorders and more.

Many children experience a high level of anxiety and may also have difficulty maintaining focus. Present interventions do not appear effective enough. Creating this protocol and device and fostering intention is a method of teaching children how to self-regulate. Observation in early school grades suggests existing classroom interventions do not appear to be sufficiently helpful in and of themselves (including weighted blankets etc. used for children on the autism spectrum). In addition to diminishing the “anxiety in the moment,” CJT has the intention of teaching the individual how to remember to “self-regulate” for future recall.

Importance would be placed on the individual using it being totally in control. The individual using it would be completely cognizant that he/she has choice and it feels right to them. CJT wants not just squeeze but the feeling of strong emotional stability with more intention to it.

Utilizing visualization and imagery would enhance the “centering,” strong feeling of emotional stability. Creating and incorporating “visualization” into the protocol would strengthen the intervention.

Somatic feeling of sensing strong emotional stability through deep pressure and weight, associated with visualization of that strong core and the positive thoughts and arousal created by a positive experience/activity can be tagged together in a mnemonic way that can be more readily recalled. In order for this to occur, the individual who understands and has mastered using the five steps, needs to want to self-regulate, have the intention to feel grounded, and the ability to recall the “feeling” of the protocol in order to succeed in returning to homeostasis. Hopefully the protocol can become integrated into our neuronal system and become an innate response resulting in improved self-regulation.

Self-regulation/feeling grounded, occurs while utilizing the protocol and can also occur with recall.

The individual, having mastered the protocol, inherently knows she can lessen anxiety and maintain strong, self-confident emotional stability.

Due to feeling “grounded”/“centered” and in a homeostatic state, through the use of the protocol, the individual experiences greater focus and increased self-confidence.

The Protocol incorporates the use of a Device. The Device is a centering jacket. All five steps of the protocol must be utilized in the manner in which they were intended; no one step can usurp the rest.

Overview of the steps:

Step one: Put the centering Jacket on. (May add aromatherapy)

Step two: Take a couple of “cleansing breaths.”

Step three: Use centering imagery.

Step four: Engage in an enjoyable, positive activity (arouses positive feelings.)

Engage in activity for approximately fifteen minutes with Jacket on, then remove Jacket and continue with activity for another fifteen minutes.

Step five: Reflection.

The Device:

The Device is a “Centering Jacket;” a vest that works through the use of (i) weighted shoulders that may feel like hands, (for adults the weight can vary per shoulder from 0.5 pounds to over 2 pounds,) and (ii) compression. The compression is to be created just at and above the waist. The compression should be firm but comfortable (depending on each individual's preference for amount of pressure.) It is to be applied for at least ten minutes but not more than an hour before taking the jacket off. The self-creation of the deep pressure/compression, while wearing the Jacket, appears to be an essential component in remembering the feeling. The individual puts the Centering Jacket on and then inflates it. In conjunction with the weighted shoulders, the compression fosters the “strong emotional stability” that appears to be remembered for an extended period of time.

The Five Steps:

1. Put the Centering Jacket on. Sit in a straight back chair with your feet on the ground, and adjust the Jacket so that it is comfortable. Place the belt around your “middle” where the compression will occur. Inflate compression by simultaneously squeezing both bulbs until experiencing a comfortable, significant degree of compression.

a. Rules and functioning of Jacket

i. Must be able to put Jacket on and take it off by oneself.

ii. Not intended for use with young children or individuals unable to put Jacket on and take it off themselves.

iii. To inflate compression, turn thumbscrews (next to bulbs) clockwise to close the deflation valve. Gently squeeze both bulbs simultaneously to level of comfort, where one feels significant pressure (e.g., pressure on the torso or shoulders).

iv. If, at any time, the individual using the device and protocol feels uncomfortable: lightheaded, dizzy or short of breath, he/she should immediately remove the Jacket.

v. Use this Jacket only as intended.

vi. The Centering Jacket is not to be worn for greater than one hour per day. It is not to be worn while actively working on Trauma with a Therapist or if experiencing any new Trauma (e.g. immediately following a car accident or a mugging.)

vii. The instructions presented here are in no way intended as a substitute for medical counseling.

viii. Deflate Jacket after step five. To deflate, turn thumbscrews to the left, allowing the Jacket to deflate gradually.

b. Add aromatherapy to pocket (optional.)

2. Take a couple of “cleansing breaths”

3. Use Centering Imagery to facilitate “centering.”

a. Examples: A beam of warm, yellow light coming down from head to toe; warm light beam coming up from feet to head and beyond; imagine your feet on a piece of granite; focus on breath perhaps while looking at a picture in the room or imagining a safe place. (Imagery is to be individualized according to preference/need; i.e.: if the individual has a history of trauma or depression, the “visualization” can be modified to meet their needs.)

4. Engaging in an enjoyable activity.

a. After a few minutes of using imagery (or individual's comfort level of time) move to involve self in activity of own choosing. (It is essential that the participant feel positively toward this “activity.”)

i. Activity must be engaging of mind and perhaps body as well. This may include listening or singing along to music, playing a musical instrument, drawing or arts and crafts work, reading, cooking, writing and sewing as well as others.

ii. Focus mind on activity, trying to stay in the “present”. If your mind wanders, gently remind yourself to return to involving yourself in the activity. Accept “where one is at” with a non-judgmental attitude of just returning to involvement in the activity.

b. After a period of time agreed upon by individual in consultation with therapist (at least ten minutes, perhaps 15-30 minutes) remove Jacket and continue activity for perhaps another 15-30 minutes.

i. When you remove the Jacket, put it aside, to deflate when the entire protocol is complete.

5. Reflection

a. Take a moment to reflect on the imagery, activity and body sensations. Feel free to take a deep cleansing breath.

Upon completion of the five steps of the Protocol, gently deflate the Centering Jacket by turning the thumbscrews to the left, gently releasing the air. Store the Jacket by laying it flat on a surface.

We have often heard the metaphors of having a “broken heart” or being yelled at and “falling to pieces. ” Whether one experiences anxiety, trauma, inability to focus, or insecurity, perhaps this Centering Jacket and Protocol hold the secrets to the “glue” that can “hold” the individual or facilitate putting the client back together; i.e.: the ability to master self-regulation.

For those who have experienced trauma and anxiety in their lives, the ability to take care of oneself with body/mind and spirit wellness may be increased through a Therapy that fosters homeostasis while incorporating many different theories from neuroscience, psychoanalytic therapy, Cognitive Behavior Therapy, imagery, mindfulness, somatic expressive therapy, massage therapy and more.

Step One, the Centering Jacket:

The moderate pressure, created by the compression, may be stimulating dermal and/or subdermal pressure receptors that are innervated by vagal afferent fibers which ultimately may implicate the autonomic nervous system resulting in a parasympathetic nervous system response. (1,2,3.) Parasympathetic activity may promote increased alertness and increased relaxation. Massage Therapy, in particular deep pressure touch, has proven to reduce stress (cortisol reduction) and activate serotonin and dopamine. (4.) Perhaps the Centering Jacket can also significantly impact human biochemistry through a method of creating compression that is self-applied and self-regulated. Furthermore, the shoulder weight and the compression at the waist give a feeling of being held or “contained” which fosters a feeling of being in greater control. The use of Centering Imagery/visualization (Step Three of Protocol) also contributes to feeling more in control. Feeling in greater control is known to diminish anxiety and increase performance (8.) The ability to remember/recall the feeling created by the Jacket for an extended period of time may result in the ability to utilize this mind/body connection to reestablish and maintain the individual's homeostasis for present use and future recall.

The following studies give support to the theories set out above and the potential effectiveness of the Centering Jacket.

Moderate Pressure Massage Elicits A Parasympathetic Nervous System Response by M. Diego and T. Field (1). In this study, a 15-minute massage therapy procedure using moderate and light pressure stroking and squeezing elicited two distinct responses. The moderate pressure showed a parasympathetic nervous system response with a high frequency component of heart rate variability suggesting increased vagal efferent activity. Light pressure increased the sympathetic nervous system response characterized by decreased HF and increased LF/HF as opposed to the results of the moderate pressure impacting the parasympathetic nervous system resulting in increased relaxation and diminished anxiety. “The effects of massage therapy on various psychological and neuroendocrine factors may be mediated by the stimulation of dermal and/or subdermal pressure receptors that are innervated by vagal afferent fibers, which ultimately project to structures involved in ANS regulation.” The PNS response supported several other studies which were characterized by decreased heart rate (e.g. (i) Diego et al, 2004; Kubsch, Neveau, & Vandertie, 2000), lower blood pressure (ii) Ahles et al., 1999; Kubsch et al., 2000), and (iii) reduced stress hormone (cortisol) levels (Field et al., 2004a; Kim, Cho, Woo & Kim, 2001)).

The moderate pressure of the compression of the Centering Jacket and the weight on the shoulders, as per the above study, may impact the parasympathetic nervous system, thereby reducing stress/anxiety.

Massage Therapy Reduces Anxiety and Enhances EEG Pattern of Alertness and Math Computations, T. Field, G. Ironson et al. (2) supports CJT's theory that the Jacket and Protocol create a feeling of being “grounded” with increased attention/focus and an increased ability to manage stress. “The superior performance of the massage therapy group might relate to the tactile and pressure stimulation. Tactile and pressure stimulation, in addition to enhancing the EEG patterns of alertness and math computations in this study, have been noted to enhance parasympathetic activity (elevated vagal tone) which is characteristic of a more relaxed, alert state during which cognitive performance improves (Field et al, 1992).

Moderate Pressure is Essential for Massage Therapy Effects, T. Field, M. Diego et. al., (3) continues to support the use of significant compression in the Centering Jacket as the article notes “massage therapy promotes relaxation and reduced stress, but only if it involves the use of moderate pressure versus light pressure. (Diego & Field, 2008; Diego, Field, Sanders, & Hernandez-Reif, 2004.)” This study, regarding moderate pressure, is supported by the previously mentioned articles. It indicates that moderate pressure (not light pressure) is the effective mechanism to promote relaxation and reduce stress. The study suggested that the stimulation of dermal pressure receptors elicited an increase in vagal activity which, supported by several other studies by their group, indicated that “moderate pressure massage therapy elicits a PNS response.”

Cortisol Decreases and Serotonin and Dopamine Increase Following Massage Therapy, T. Field, M. Hernandez-Reif, M. Diego, S. Schanberg and C. Kuhn (4) is an additional study supporting the positive effects of massage therapy on biochemistry. Perhaps, the Centering Jacket can replicate these findings.

Temple Grandin's article, Calming Effects of Deep Touch Pressure in Patients with Autistic Disorder, College Students, and Animals, states that, anecdotally, with the use of the “Squeeze Machine,” there is clinical value in children with autistic disorder and probably with children with ADHD in reducing anxiety and nervousness. (5)

Swaddling. A Systematic Review, reports that, in general, swaddled infants arouse less and sleep longer. “When compared with massage, excessively crying infants cried less when swaddled, and swaddling can soothe pain in infants.” (6)

The clinical application of deep touch pressure with a man with autism presenting with severe anxiety and challenging behavior, by S. Blairs , S. Slater, and D. J. Hare (7) is an empirical study of the use of deep pressure touch. The Centering Jacket may be related to a self-applied, self-controlled restraining, deep pressure touch device. This study made deep pressure touch a part of this man's scheduled protocol and it appeared to reduce the levels of restraint and medication that this man needed. This study used a ‘tuck-in’ procedure to provide the deep pressure touch. It was observed that there were reductions in blood pressure, pulse rate, respiration rate and body temperature pointing to the efficacy of the intervention.

It is reported that feeling out of control emerged as a major theme, with cognitive anxiety being more dominant. Walker et. al. discuss the personal variables of anxiety including the perception of control, self-confidence and the use of psychological skills which help to determine how the athletes interpret their anxiety symptoms. The result of the study was to recommend to dancers education on prevention or handling of anxiety symptoms to help them feel more in control. “Dancers who feel in control of anxiety, perhaps due to increased self-confidence, may interpret anxiety as predominantly facilitative.”

The Centering Jacket appears to facilitate a feeling of being “grounded” and more in control. Connecting the deep pressure of Step One with the other Steps of the Protocol appears to diminish anxiety, and increase focus and self-confidence, facilitating a greater sense of being in control and supporting mastery, thereby diminishing performance anxiety for athletes, (dancers etc.)

Taking a look at other hypotheses, Joseph LeDoux reports “connections between neurons, known as synapses, are the main channels of information flow and storage in the brain. Most of what the brain does is accomplished by synaptic transmission between neurons, and by calling upon the information encoded by past transmission across synapses.” (9) The Jacket creates the feeling of being “held” or “contained.” It is possible that this feeling may be reminiscent of early childhood experiences and/or neuronal encoding from an early age. Does the “holding” and/or hugging of infants early in life encode a synaptic transmission between neurons to remember the feeling of comfort and/or control experienced by being held? Does the device, the feeling/compression of being contained or held, facilitate GABA cell transmission, thereby reducing fear and anxiety? Do they enhance inhibition in the amygdala? Or, possibly, it is the compression that is decreasing inflammation that was caused by the trauma? Salim, Chugh and Asghey report an interaction between the immune system and the CNS (Central Nervous System). They are studying the role of cytokines in regulating neurotransmitters. (10)

One can also look at the psychological aspects along with the neurobiological aspects of the Jacket and Protocol. One can ask if it is the positive memory of being held in the initial maternal-child environment that is being remembered? Is the “sense of self” created by the weight and compression facilitating the remembering of the early childhood experience of being held and soothed? Winnicott and, more recently, Allan Shore, discuss the relationship between neurobiology and psychology, specifically the significance of “good enough mothering” and the importance of early attachment. Although D. W. Winnicott's description of the “holding environment” is interpersonal and the essence is in the relationship, Winnicott did report that when a child is deprived of an environmental control (12) the child completely loses his identity. When controls are re-imposed, the child begins to regain confidence and re-establish himself. Thus the Centering Jacket's creation of a feeling of being in control may increase the individual's self-confidence. The question arises: are Winnicott and LeDoux, one working in the psychiatric realm and the other in neuro-biology, able to intersect and the interaction between the two beliefs both retain relevancy? We believe so. Whether or not we are discussing very early holding, where the infant's sense of inner and outer self are just developing, or the 12-24 month old who mutually hugs, many psychological and neuro-biological systems are at work developmentally. Could the somatic experience of the Jacket be, in part, replicating a positive experience that had a neuronal impact? The answers are unknown at present but it may be a combination of some or all of the above. What is known is that feeling “in control” decreases anxiety and increases self-confidence. (Further discussed under Step Three.) Allan Shore (13) also discusses tactile and kinesthetic sensations guiding early attachment behavior as well as helping to regulate the infant's behavior and physiology. In his book, Affect Regulation and the Origin of the Self, Shore argues that the early social environment, mediated by the primary caregiver, influences the evolution of the structures of the infant's brain; this has a direct impact on the infant's ability to self-regulate emotions and manage stress. Once again, is the Centering Jacket bringing forth those early memories to create new strength to facilitate self-regulation?

The referenced studies on deep pressure touch and massage provide support for the proposition that the Parasympathetic Nervous System responds to the deep pressure, resulting in increased relaxation and decreased stress/anxiety. Utilizing data from Massage Therapy is relevant to CJT because they are both utilizing deep pressure touch and stimulation. With the Massage Therapy, a therapist applies the pressure; with CJT, the pressure/compression is self-applied and the compression occurs while the individual is wearing the Jacket. The one person study on deep pressure touch with a man with Autism supports CJT's theory that self-regulated, self-applied restraint or pressure can have a positive impact on decreasing stress and fostering a return to homeostasis. The study of Temple Grandin's hug machine showed “a significant reduction in tension and anxiety for the experimental group as well as a decrease in galvanic skin response measures after deep pressure.” (5) CJT creates an intention for the usage of the Centering Jacket as well as facilitating “mindfulness” and a mind/body connection.

The Centering Jacket, which appears to create the feeling of being “held”, feeling in control, and a sense of well-being, anchored to the next steps, including Cleansing Breaths, Centering Imagery and success and/or positive feelings from engaging in a pleasing activity, is an attempt to improve the connection between the unconscious and the conscious mind/body connection and through the use of CJT and our knowledge of the neuroplasticity of the brain, improve the individual's ability to maintain homeostasis. Ledoux points out that learning is a lifelong process. Mark Johnson states: (11) “Indeed much of the self is learned . . . memories created are dependent on things we've learned before.” The five steps, playing tag with each other, may be creating a blueprint for changing the brain, in a positive way, to assist in maintaining homeostasis, greater self-confidence and greater stability in functioning. Hopefully, over time, the memory of the Protocol will become automatic.

The Centering Jacket creates both the physiological stimulus, and the somatic experience that facilitate putting the individual in a mindful, homeostatic state, resulting in feeling “grounded.” The Jacket, coupled with the following four steps, appears to reinforce a “mindful state” and engage the individual in automatically being “in the present” while having a “positive” experience, resulting in improved self-regulation. Pat Ogden (14) states “mindfulness is the key to clients becoming aware of internal sensory-motor reactions which increase the capacity for self-regulation.” All five steps of CJT encourage the individual to be in the “present”, creating an awareness of both body and mind, thereby supporting the intention and facilitating the mastery of the Protocol. The first step, of putting the Jacket on, experiencing the weight on the shoulders, and creating the compression, leads us into Step Two.

Step Two: Take two cleansing breaths.

The purpose of the cleansing breaths is really to create “intention” for the task at hand and help facilitate maintaining a mindful state in the here and now. Cleansing breaths also foster relaxation. Attention during memory retrieval enhances future remembering by N. Dudukovic, S. DuBrow et al. (15) indicates performance is much improved when one uses their full, undivided attention. “Attention is not only necessary during memory encoding, (e.g. Baddeley et al 1984, Craik et al 1996, Mulligan, 1998) it is also important during memory retrieval to endure future remembering.” (15.) The cleansing breaths promote the attention and focus necessary for the protocols success.

CJT believes one may return to homeostasis by using this therapeutic process, which appears to work without having to over think and train. By merely using this body/mind/spirit approach, one may automatically become “self-regulated,” and fully present in the moment. Creating intention is a step towards mastery.

Step Three: Using Centering Imagery:

Centering imagery may facilitate self-regulation and foster being relaxed, and self-confident, in the present moment. Furthermore, according to Bellaruth Naparstek, research findings support imagery and guided imagery's use due to a positive impact on health, creativity and performance. (Staying Well with Guided Imagery, 1994.) (16) Naparstek discusses three guiding principles: the mind-body connection, the altered state and the locus of control. She indicates that when the individual feels a sense of being in control with the use of imagery, they feel better about themselves and perform better. “Feeling in control is associated with higher optimism, self-esteem, and ability to tolerate pain, ambiguity and stress.” (Naparstek 2000, Staying well with guided imagery, 1994). The Military is presently utilizing, with success, Guided Imagery to treat Post-Traumatic Stress Disorder. Using centering imagery reinforces the intention of the Protocol and the strong feeling of the “core” created by the Centering Jacket.

Step four, engaging in a positive activity:

It is essential that the activity is pleasing and positive so that the association of feeling grounded and centered is attached to the explicit memory of a positive experience. Pleasing activities promote a “mindful” state; they encourage the individual to be in the present and focused while having an enjoyable time. It encourages positive affect and positive thinking. The activity may help anchor the individual in remembering the positive, relaxed and core strengthened feeling of creativity, success and a sense of well-being. The “feel-good” experience, perhaps the creativity, and the feeling of being held and/or contained by the Jacket can only add to the sense of self, integration and self-regulation.

The Self-Imagination Effect: Benefits of a Self-Referential Encoding Strategy on Cued Recall in Memory-Impaired Individuals with Neurological Damage, by M. Grilli and E. Glisky (17) indicates that self-imagining enhanced cued recall more than visual imagery, semantic elaboration and other-imagining. Self-imagination is examining something from a personal perspective. This protocol's fives steps will hopefully prove to show an association with each other. The idea of engaging in an activity that one feels positive about, is another way of using “self-imagination” and something unique to that individual to tag to the feeling of being grounded that has been created by the Jacket.

Instructions in this Protocol to be in the present, in a “mindful” state, and gently bringing oneself back to the present in a non-judgmental way, facilitate homeostasis in an explicit way. According to Dr. Sara Lazar at Harvard Medical School and Dr. Ulrich Ott at Justus Liebig University in Germany, there are four ways mindfulness works: attention regulation, body awareness, emotion regulation and sense of self. CJT incorporates the four ways mindfulness works. Attention regulation trains the mind to overcome distraction, which can lead to feeling centered. Attention is the focus on the somatic and the activity at the present moment. Body awareness is facilitated by the deep pressure and weight of the jacket. CJT teaches emotion regulation. Engaging in an activity where one experiences a positive feeling either from enjoyment and/or creativity, and the individual has an awareness of those feelings in conjunction with the body awareness from the Jacket, appears to result in a greater sense of self.

Engaging in a pleasing activity, first with the Centering Jacket on, with the weight and compression, and then continuing the activity, having taken the Jacket off, is giving the individual two opportunities: 1) to see that the feeling of being held or contained continues once the Jacket is removed, 2) being in the “present”, a mindful state, while enjoying an activity, reinforces that the individual can remember the feeling of being grounded while engaging in something fun that hopefully sparks a positive feeling.

The Protocol requires the individual to be “mindful” and gently, in a non-judgmental way, be aware to stay in the present with attention on the positive activity. CJT is not a mindfulness meditation/practice. It incorporates the need to be in the present moment but does not divert attention away from internal and external emotional situations; it wants to create an awareness of the internal and external experience. CJT wants the individual to be aware of the positive arousal created by the activity and to embrace the “good” feeling. The ability to recall the positive experience can be an anchor to the strength to cope with the present, resulting in diminished anxiety and a return to homeostasis.

Step five, engaging in reflection of the experience:

Step five is an opportunity to integrate the experience of CJT. The different components, separately and together, may affect individuals differently. Those that are visual may find the imagery the “anchor.” Others may find the compression and/or weight of the Jacket and the impact on the body the most important anchor. Some may find the aromatherapy to be the key, while other individuals may find the creativity and/or the success of engaging in a positive activity so powerful after the initial steps of CJT that the activity will be their anchor for future ability to readily regulate themselves.

The protocol stipulates not to use the Centering Jacket while actively working on Trauma or experiencing new Trauma (e.g.: being in a car accident and the immediate aftermath or immediately following being mugged, etc.) The reasoning is twofold: #1) In Somatic Expressive Therapy (SE) developed by Peter A. Levine, PhD. Dr. Levine's research shows the necessity of bodily/somatically expressing and releasing the stress of trauma. (In an Unspoken Voice, Peter A. Levine. [18]) Utilizing the Jacket while actively working on Trauma would not allow that release of energy. #2) CJT's protocol anchors each step to each other; it is important to connect a relaxed state, feeling contained, and centering imagery with a positive activity thereby not perpetrating further trauma.

In Norman Doidge's book, The Brain That Changes Itself, he states that Eric Kandel directly attests to the neuroplasticity of the brain. “Kandel was the first to show that as we learn, our individual neurons alter their structure and strengthen the synaptic connections between them. He was also first to demonstrate that when we form long-term memories, neurons change their anatomical shape and increase the number of synaptic connections they have to other neurons.” (19) It is hoped that CJT can facilitate long-term changes in the brain that result in ongoing, improved homeostasis as well as increased well-being.

This last step of reflection solidifies the intention of the Protocol; it reinforces the thoughts, feelings and connection of all Five Steps.

Summary:

In conclusion, Centering Jacket Therapy may facilitate reduced stress/anxiety and increased self-confidence. It may educate the individual in the understanding and experiencing of the body-mind connection. CJT may promote homeostasis and will hopefully prove mastery of the ability to self-regulate.

When the individual becomes aware of excess anxiety, dissociation, inability or difficulty concentrating, and/or difficulty organizing oneself, utilizing the Protocol, including the Jacket, or if the Jacket is unavailable, utilizing “recall,” by thinking about one or two of the steps and the others should follow, as if a mnemonic device. Hopefully, through a clinical trial, it will be proved that utilizing this Protocol, and/or recalling it, results in the ability to self-regulate.

When the individual focuses on each step with the intention of connecting all Five Steps, the expectation then becomes the positive outcome, which can be defined as facilitating self-regulation. Simultaneously decreasing anxiety while engaging in a positive activity, with intention, increases positive affect and creates mastery of the ability to self-regulate: Mastery Accomplished.

The Jacket and the Protocol are a learning and healing experience and can be beneficial for this generation and future generations.

What is claimed is:

1. A wearable device for treating or preventing emotionally based dysregulation in a subject comprising an expandable device covering at least a portion of a torso of a subject comprising: (a) one or more compartments capable of receiving a weighted object; (b) one or more expandable compartments capable of being inflated to apply pressure to a specific body region of the torso of the subject when expanded; c) a mechanism for expanding said individually expandable compartments; and d) a control unit capable of operating said individually expandable units in a sequence of expansions and contractions so as to r for treating or preventing emotionally based dysregulation.

2. The wearable device of claim 1, further comprising one or more sensing unit for measuring at least one physiological parameter of the subject.

3. The wearable device of claim 2, wherein said sensing unit includes at least one sensor selected from the group consisting of a heart rate sensor, a body temperature sensor, a skin moisture sensor, a muscle activity sensor, a blood pressure sensor, a respiratory sensor and a brain activity sensor.

4. The wearable device of claim 3, wherein the expandable units apply one or more expansions and contractions determined by physiological parameter data values of the individual monitored by said sensing unit.

5. The system of claim 1, wherein said wearable device is configured as a vest.

6. The system of claim 1, wherein said mechanism for expanding includes a manual air pump.

7. A method for treating or preventing emotionally based dysregulation in a subject comprising providing a subject with a wearable device comprising an expandable device covering at least a portion of a torso of a subject comprising:
(a) one or more compartments capable of receiving a weighted object; (b) one or more expandable compartments capable of being inflated to apply pressure to a specific body region of the torso of the subject when expanded; c) a mechanism for expanding said individually expandable compartments; and d) a control unit capable of operating said individually expandable units in a sequence of expansions and contractions so as to treat or prevent emotionally based dysregulation.

8. The method of claim 7, further comprising one or more sensing unit for measuring at least one physiological parameter of the subject.

9. The method of claim 7, wherein said sensing unit includes at least one sensor selected from the group consisting of a heart rate sensor, a body temperature sensor, a skin moisture sensor, a muscle activity sensor, a blood pressure sensor, a respiratory sensor and a brain activity sensor.

10. The method of claim 8, wherein the expandable units apply one or more expansions and contractions determined by physiological parameter data values of the individual monitored by said sensing unit.

11. The method of claim 7, wherein said wearable device is configured as a vest.

12. The method of claim 7, wherein said mechanism for expanding includes a manual air pump.
